(19) United States
(12) Patent Application Publication
(10) Pub. No.: US 2026/0022385 A1
(43) Pub. Date: Jan. 22, 2026

(54) DNA APTAMERS THAT BIND TO ALGINATE GELS

(71) Applicant: Saint Louis University, St. Louis, MO (US)

(72) Inventors: Dana Ann Baum, Collinsville, IL (US); Ali Parvez, Richmond Heights, MO (US); Damon Michael Osborne, St. Louis, MO (US)

(21) Appl. No.: 19/274,228

(22) Filed: Jul. 18, 2025

Related U.S. Application Data
(60) Provisional application No. 63/672,873, filed on Jul. 18, 2024.

(51) Int. Cl. C12N 15/115; A61K 47/54; A61K 47/64; A61K 47/69; G01N 33/53

(52) U.S. Cl. CPC C12N 15/115; A61K 47/549; A61K 47/643; A61K 47/6903; G01N 33/5308; C12N 2310/16; C12N 2310/3513; G01N 2333/765

(57) ABSTRACT

Disclosed are alginate-specific deoxyribonucleic acid (DNA) aptamers that specifically bind to alginate hydrogels and alginate hydrogels containing alginate-specific DNA aptamers. The alginate-specific DNA aptamers display slower diffusion out of alginate hydrogels and function as anchors for other biomolecules in a wide range of applications.

### CROSS REFERENCE TO RELATED APPLICATIONS

This application claims priority to and benefit of U.S. Provisional Patent Application Ser. No. 63/672,873, filed on Jul. 18, 2024, the disclosure of which is incorporated herein by reference in its entirety.

### INCORPORATION OF SEQUENCE LISTING

A computer readable form of a Sequence Listing containing the file named “SLU24-017. xml”, created on Jul. 18, 2024 which is 9,153 bytes in size as measured in MICROSOFT WINDOWS® EXPLORER), is provided herein and is herein incorporated by reference. This Sequence Listing consists of SEQ ID NOs: 1-10.

### BACKGROUND OF THE DISCLOSURE

The present disclosure relates generally therapeutic delivery via hydrogel compositions. In particular, the present disclosure is directed to deoxyribonucleic acid (DNA) aptamers that specifically bind to alginate hydrogels and alginate hydrogels containing DNA aptamers. The DNA aptamers display slower diffusion out of alginate hydrogels and function as anchors for other biomolecules in a wide range of applications.

Hydrogels are becoming common in the clinical treatment of wounds. Alginate and chitosan hydrogels, which are biocompatible, non-toxic materials with the inherent ability to retain water. Alginate, a naturally occurring polysaccharide extracted from marine brown algae, is the sodium salt of alginic acid. This linear copolymer is composed of (1, 4)-linked β-D-mannuronic acid (M) and α-L-guluronic acid (G) residues. Alginate forms a hydrogel in the presence of divalent metal ions, such as calcium. These properties are useful in developing newer wound dressings. For example, a widely available 3M TEGADERM™ alginate dressing is made from calcium alginate fibers and readily forms a robust healing environment in the presence of wound exudates. This material serves as a stable aseptic environment and has been shown to possess wound-healing properties. These alginate hydrogels are also very porous, so using these gels to encapsulate biomolecules or therapeutic agents could be challenging due to relatively rapid diffusion out of the gel.

Several approaches have been explored to control the release of biomolecules from alginate hydrogels. One of the most common methods is to directly functionalize the hydrogel with the therapeutic during the crosslinking for gelation. The therapeutic is then released slowly over time as the gel degrades. In another method, 18β-glycyrrhetinic acid (GA) and bovine serum albumin (BSA) trapped within poly (D,L lactic) microspheres were dispersed in a hydrogel during gelation. The diffusion profile of this system revealed rapid BSA release but a slow and sustained release of GA. Other approaches employ nanoparticles to control the gelation properties of alginate hydrogels. Although these approaches offer improved drug release over control methods, these methods require specialized fabrication to provide very tight control of the morphology and composition of the alginate hydrogels, making the sample preparation for drug delivery complicated and tedious. A robust system that can be applied to a wide variety of available therapeutics would be advantageous.

Hydrogel properties have also been controlled by incorporation of aptamers. Aptamers are short, synthetic single-stranded (ss) DNA or RNA sequences that display affinity and specificity to various targets, including proteins, chemical compounds, cells, and micro-organisms. Aptamers are identified through an iterative in vitro process known as Systematic Evolution of Ligands by Exponential Enrichment (SELEX) or in vitro selection. Aptamer-functionalized hydrogels incorporate these short DNA and RNA sequences via modification of the hydrogel during the crosslinking. The gelation and/or degradation of the gel is controlled by introducing the aptamer target, which produces a structural change in the aptamers and changes the morphology of the hydrogel. However, these methods also require direct modification of the hydrogel. A modular approach in which diffusion of the therapeutic can be impacted without directly changing the hydrogel properties would expand the therapeutic potential of hydrogel materials.

Drug binding to Human Serum Albumin (HSA) plays a pivotal role in determining the pharmacokinetic and pharmacodynamic profiles of therapeutic compounds. HSA, the most abundant protein in plasma, functions as a depot and carrier for a range of endogenous and exogenous substances, including many drugs. The interaction between drugs and HSA affects their distribution, clearance, and ultimately their efficacy. The primary impact of drug-HSA binding is on the pharmacokinetics of the drug, including its distribution, metabolism, and excretion. Binding affinity affects the free concentration of a drug, thereby influencing its biological activity. Drugs bound to HSA are in a reversible, non-active state, whereas free, unbound drugs are biologically active. This binding can also prevent drugs from crossing the cell membranes, limiting their distribution to various tissues. HSA has multiple binding sites, particularly within subdomains IIA and IIIA, which accommodate a variety of drugs. The interaction is primarily driven by hydrophobic forces, although other types of interactions, such as electrostatic interactions, may also be involved depending on the drug. The binding process can be characterized by either enthalpy-driven or entropy-driven mechanisms.

Accordingly, there exists a need for new hydrogels with controllable diffusion of biomolecules for therapeutic delivery. Further, understanding the binding of drugs to HSA can inform therapeutic decision-making and drug design. For instance, the competitive binding of multiple drugs to HSA can lead to drug-drug interactions, potentially altering the efficacy or increasing the toxicity of the medications involved. The displacement of a bound drug by another drug is clinically relevant, as it can lead to increased concentrations of the free, active form of the displaced drug, thereby enhancing its effects or toxicity.

The present disclosure provides a solution for these needs by providing alginate-specific DNA aptamers and alginate hydrogels incorporating the alginate-specific DNA aptamers. The alginate-specific DNA aptamers display slower diffusion out of the alginate hydrogels and can serve as anchors for other biomolecules in a wide range of applications. The alginate-specific aptamers enable immobilization of HSA within a stable alginate hydrogel environment allowing for testing binding of various drugs to aptamer-immobilized HSA. Not only can measurements be made on the amount of drug captured by the bound HSA, but if desired, the HSA could later be released to isolate the HSA-drug complexes.

### BRIEF DESCRIPTION OF THE DISCLOSURE

In one aspect, the present disclosure is directed to an alginate-specific deoxyribonucleic acid (DNA) aptamer.

In another aspect, the present disclosure is directed to an alginate hydrogel comprising alginate and an alginate-specific DNA aptamer.

In another aspect, the present disclosure is directed to a method for selecting an alginate-specific DNA aptamer. The method comprises (a) reconstituting a DNA oligonucleotide mixture in an alginate solution; (b) initiating gelation of the alginate solution to form an alginate hydrogel including the DNA oligonucleotide mixture; (c) incubating the alginate hydrogel in a selection buffer; (d) collecting the selection buffer; (e) degrading the alginate hydrogel to form a degradation solution; (f) treating the degradation solution with an alginate lyase solution; (g) collecting the alginate-specific DNA aptamers; and (h) amplifying the alginate-specific DNA aptamers.

In another aspect, the present disclosure is directed to a method for delivering to a subject in need thereof a biomolecule in an alginate hydrogel. The method comprises: providing to a subject in need thereof an alginate hydrogel, the alginate hydrogel having a biomolecule-alginate-specific DNA aptamer conjugate, wherein the biomolecule-alginate-specific DNA aptamer conjugate specifically binds to alginate by the alginate-specific DNA aptamer and wherein the biomolecule-alginate-specific DNA aptamer conjugate is released from the alginate hydrogel to deliver the biomolecule.

In another aspect, the present disclosure is directed to a method of analyzing binding of a small molecule drug to human serum albumin, the method comprising contacting a small molecule drug with an alginate hydrogel, the alginate hydrogel comprising an alginate-specific DNA aptamer conjugated with human serum albumin; washing the alginate hydrogel with a wash solution; and analyzing the wash solution to determine binding of the small molecule drug to the human serum albumin.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The disclosure will be better understood, and features, aspects and advantages other than those set forth above will become apparent when consideration is given to the following detailed description thereof. Such detailed description makes reference to the following drawings, wherein:

FIG. 1 depicts a SELEX approach for isolating alginate-specific aptamers.

FIG. 2 depicts disc diffusion and spin column assays for assessing alginate binding.

FIG. 3A is a graph depicting aptamer elution during the selection process.

FIG. 3B is a graph depicting aptamer elution of sequences from round 7 of the selection process using the disc diffusion.

FIGS. 4A and 4B are graphs depicting disc diffusion of 7CF and 7CH sequences. For the 7CF clones, 7CF13, 7CF26, and 7CF27 were retained longer in the hydrogel than controls and produced a strong spike when the hydrogel was degraded completely (FIG. 4A). For the 7CH clones, 7CH31 produced the strong spike when the hydrogel was degraded (FIG. 4B).

FIG. 5 depicts the binding profile of aptamers 7CF13 and 7CH31in the spin column elution approach used during SELEX.

FIGS. 6A-6C are graphs depicting aptamer binding to 0.5% (FIG. 6A), 1% (FIG. 6B, or 2% (FIG. 6C) alginate hydrogels.

FIGS. 7A and 7B are graphs depicting disc diffusion of aptamers from 1% alginate hydrogels (FIG. 7A) and agarose hydrogels (FIG. 7B).

FIGS. 8A-8C depict aptamer binding to pre-formed alginate hydrogels. FIG. 8A is a graph depicting aptamer elution from pre-formed alginate hydrogels for 24 hours followed by alginate hydrogel degradation. FIG. 8B is a graph depicting aptamer elution from pre-formed alginate hydrogels for 7 days followed by alginate hydrogel degradation. FIG. 8C are photographs of alginate hydrogels showing aptamer nucleation sites in the alginate hydrogels.

FIGS. 9A-9D are graphs depicting diffusion of BSA-aptamer conjugates from alginate hydrogels. FIG. 9A shows diffusion of encapsulated BSA conjugated to control oligonucleotides or alginate-specific aptamers. FIGS. 9B and 9C show the ability of the aptamer-BSA conjugates to enter and then be retained by preformed alginate hydrogel discs using two different percentages of alginate hydrogel discs. FIG. 9D depicts retention of aptamer-BSA conjugates versus unconjugated BSA in alginate hydrogels within spin columns.

FIG. 10 is a graph depicting the HSA binding profile for Atorvastin, Propranolol, and Gabapentin in alginate gels containing aptamer-HSA conjugates. Differences emerged in percentage elution of drugs through the different wash steps.

### DETAILED DESCRIPTION

Unless defined otherwise, all technical and scientific terms used herein have the same meaning as commonly understood by one of ordinary skill in the art to which the disclosure belongs.

In one aspect, the present disclosure is directed to an alginate-specific DNA aptamer.

Particularly suitable alginate-specific DNA aptamers include SEQ ID NO:1 and SEQ ID NO:2.

Suitably, the alginate-specific DNA aptamers can be modified. It will be understood that possible modifications will depend on the particular application.

The alginate-specific DNA aptamers can include a primer binding site(s). Deoxyribonucleic acid (DNA) used to generate the alginate specific DNA aptamers include natural DNA nucleotides and modified nucleotides. The DNA nucleotides can provide chemical handles for attaching other molecules/labels. Backbone modifications can provide enhanced stability depending on application. The alginate-specific DNA aptamers can include 5′ and 3′ attachment of labels or for attachment to the molecules intended to be delivered from the hydrogel, and/or used in the hydrogel. A wide range of labels and modifications are commercially possible (see e.g., Glen Research, Sterling, VA, USA).

In one embodiment, the alginate-specific DNA aptamer includes serum albumin. Suitably, serum albumin includes human serum albumin and bovine serum albumin.

The alginate-specific DNA aptamer and serum albumin are conjugated using methods known in the art. One suitable method is using the coupling agent DMT-MM (4-(4,6-dimethoxy-1,3,5-triazin-2-yl)-4-methyl-morpholinium chloride). DMT-MM is used for activation of carboxylic acids, particularly for amide synthesis. Additional suitable methods involving amide synthesis are using the coupling agent DCC (dicyclohexylcarbodiimide) or coupling agent EDC (1-ethyl-3-(3-dimethylaminopropyl)carbodiimide in the presence or absence of NHS (N-hydroxysuccinimide).

In another aspect, the present disclosure is directed to an alginate hydrogel including an alginate-specific DNA aptamer.

Suitable alginate-specific DNA aptamers are described herein.

In one embodiment, the alginate hydrogel includes an alginate-specific DNA aptamer wherein the alginate-specific DNA aptamer is conjugated with serum albumin. In one embodiment, the alginate hydrogel includes an alginate-specific DNA aptamer and an alginate-specific DNA aptamer conjugated with serum albumin. Suitable serum albumin includes human serum albumin and bovine serum albumin.

Alginate hydrogel is prepared using methods known in the art. An alginate hydrogel precursor solution includes alginate that forms an alginate hydrogel upon crosslinking. Suitably, the amount of alginate in the final alginate hydrogel precursor solution ranges from about 0.05% (wt/wt) to about 10% (wt/wt). Preferably, the amount of alginate ranges from about 0.5% (wt/wt) to about 2% (wt/wt).

To prepare an alginate hydrogel including an alginate-specific DNA aptamer, the alginate-specific DNA aptamer is mixed with an alginate hydrogel precursor solution and gelation is initiated by the addition of a crosslinking agent such as calcium chloride.

The alginate hydrogel can further include a chelating gelation control reagent for controlling hydrogel formation of alginate hydrogel. Suitable chelating gelation control reagents include ethylenediaminetetraacetic acid (EDTA) and/or glucono-δ-lactone (GDL). Other suitable chelating gelation control reagents include: nitrilotriacetic acid (NTA), trans-1,2-diaminocylcohexanetetraacetic acid (DCTA), diethlyenetriaminepentaacetic acid (DTPA), and bis(aminoethyl)glycolether-N,N,N′,N′-tetraacetic acid (EGTA).

As understood by one of ordinary skill in the art, the alginate hydrogel can be stiffened by treating the hydrogel with calcium chloride.

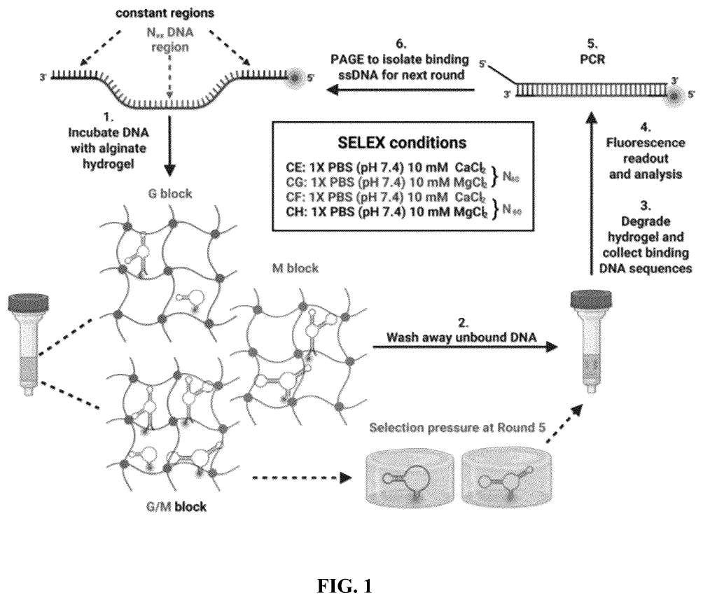

In another aspect, the present disclosure is directed to a method for selecting an alginate-specific DNA aptamer. An exemplary embodiment of the method is illustrated in FIG. 1.

The method includes (a) reconstituting a DNA oligonucleotide mixture in an alginate solution; (b) initiating gelation of the alginate solution to form an alginate hydrogel including the DNA oligonucleotide mixture; (c) incubating the alginate hydrogel in a selection buffer; (d) collecting the selection buffer; (e) degrading the alginate hydrogel to form a degradation solution; (f) treating the degradation solution with an alginate lyase solution; (g) collecting the alginate-specific DNA aptamers; and (h) amplifying the alginate-specific DNA aptamers. The method can further include repeating steps (a) through (h).

The DNA oligonucleotide mixture includes DNA oligonucleotides ranging in length from about 10 nucleotides to about 250 nucleotides, including from about 20 nucleotides to about 250 nucleotides, including from about 20 nucleotides to about 75 nucleotides, and including from about 20 nucleotides to about 70 nucleotides. The DNA oligonucleotides can further include primer binding regions. DNA oligonucleotides can be synthesized and obtained from commercial sources such as Integrated DNA Technologies (IDT, Coralville, IA). The synthesized DNA oligonucleotides can be purified using methods such as denaturing polyacrylamide gel electrophoresis (PAGE) and phenol: chloroform isoamyl alcohol (PCI) extraction.

The amount of alginate in the final alginate hydrogel composition ranges from about 0.05% (wt/wt) to about 10% (wt/wt). Preferably, the amount of alginate ranges from about 0.5% (wt/wt) to about 2% (wt/wt).

Gelation of the alginate solution containing the DNA oligonucleotide mixture is initiated and the alginate solution is placed in a spin column and allowed to gel to form an alginate hydrogel wherein the DNA oligonucleotide mixture is encapsulated by the alginate hydrogel. Gelation can be initiated as known in the art, for example, by adding NaCl, CaCl₂, and glucono-1,5-lactone. In a preferred embodiment, gelation of the alginate solution containing the DNA oligonucleotide mixture is performed in a spin column. It should be appreciated that gelation can generally be performed in any mold such as an Eppendorf, a well of a multi-well dish, and the like.

The method then includes incubating the DNA oligonucleotide hydrogel in a selection buffer. Following incubation, the selection buffer is exchanged by centrifuging the spin columns and collecting the flow-through. The buffer exchange and flow-through collection step can be repeated 1 or more times. Each exchange of the selection buffer results in obtaining alginate-specific DNA aptamers having higher affinity for alginate.

The alginate-specific DNA aptamers collected from the flow-through can be amplified. The amplified alginate-specific DNA aptamers can then be incorporated into an alginate hydrogel as described herein to further subject the amplified alginate-specific DNA aptamers for alginate-specific DNA aptamers having higher affinity for alginate binding. As described herein, each round of alginate hydrogel selection for alginate-specific DNA aptamers and amplification results in obtaining alginate-specific DNA aptamers having higher affinity for alginate.

The method further includes releasing the aptamers from the alginate hydrogel by degrading the alginate hydrogel in a hydrogel degradation buffer to form a degradation solution. A suitable hydrogel degradation buffer includes 1× PBS, 100 mM EDTA, and 2 M urea. It should be understood that degrading the alginate hydrogel need not degrade the alginate hydrogel to monomers. Other suitable methods for degrading the alginate hydrogel include incubating the alginate hydrogel in a solution of EDTA, incubating the alginate solution in phosphate buffered saline and applying heat.

The method further includes treating the degradation solution with an alginate lyase solution and collecting the alginate-specific DNA aptamers. A suitable alginate lyase solution includes 1.5 U of alginate lyase in 20 mM MES pH 4.5. After the alginate lyase treatment, the method includes removal of any incompletely degraded alginate hydrogel fragments. Suitably, the incompletely degraded alginate hydrogel fragments can be removed from the sample using an Amicon 15K centrifugal column.

The alginate-specific DNA aptamers can be collected using denaturing polyacrylamide gel electrophoresis (PAGE) and phenol:chloroform isoamyl alcohol (PCI) extraction.

In the method, alginate-specific DNA aptamers having variable affinity for alginate can be obtained following incubation of the alginate hydrogel in the selection buffer step(s), following the collection of the selection buffer steps, following the alginate hydrogel degradation step, following the alginate lyase solution treatment step and combinations thereof. During the method, alginate-specific DNA aptamers having lower affinity for alginate are obtained following incubation of the alginate hydrogel in the selection buffer step(s) and alginate-specific DNA aptamers having higher affinity for alginate are obtained following the alginate hydrogel degradation step and following the alginate lyase solution treatment step. Suitably, the method includes at least 2 rounds of the method to obtain alginate-specific DNA aptamers. The affinity of the alginate-specific DNA aptamers is improved by a least 3-fold from 2 rounds to 7 rounds of the method.

The method can further include a disc diffusion step. The disc diffusion step includes preparing an alginate solution with alginate-specific DNA aptamers; initiating gelation to form an alginate hydrogel disc with alginate-specific DNA aptamers; incubating the alginate hydrogel disc with selection buffer; exchanging the selection buffer; collecting the selection buffer; degrading the alginate hydrogel disc to form a degradation solution; and collecting the alginate-specific DNA aptamers from the selection buffer, the degradation solution, and combinations thereof. The disc diffusion step can optionally include treating the degradation solution with an alginate lyase solution and collecting the alginate-specific DNA aptamers from the alginate lyase solution. The disc diffusion step can optionally include removal of any incompletely degraded alginate hydrogel fragments after the alginate lyase treatment. The method can further include amplifying the alginate-specific DNA aptamers from the selection buffer, the degradation solution, the alginate lyase solution, and combinations thereof, to obtain amplified alginate-specific DNA aptamers.

Suitable amount of alginate in the final alginate hydrogel composition ranges from about 0.05% (wt/wt) to about 10% (wt/wt). Preferably, the amount of alginate ranges from about 0.5% (wt/wt) to about 2% (wt/wt).

The method can further include a disc diffusion step on the amplified alginate-specific DNA aptamers. The method includes preparing an alginate solution with the amplified alginate-specific DNA aptamers; initiating gelation to form an alginate hydrogel disc with the amplified alginate-specific DNA aptamers; incubating the alginate hydrogel disc with selection buffer; exchanging the selection buffer; collecting the selection buffer; degrading the alginate hydrogel disc to form a degradation solution; and collecting the amplified alginate-specific DNA aptamers from the selection buffer, the degradation solution, and combinations thereof. The disc diffusion step can optionally include treating the degradation solution with an alginate lyase solution and collecting the amplified alginate-specific DNA aptamers from the alginate lyase solution. The disc diffusion step can optionally include removal of any incompletely degraded alginate hydrogel fragments after the alginate lyase treatment. The method can further include amplifying the amplified alginate-specific DNA aptamers from the selection buffer, the degradation solution, the alginate lyase solution, and combinations thereof, to obtain amplified alginate-specific DNA aptamers. The disc diffusion step of the amplified alginate-specific DNA aptamers can be repeated for any number of desired rounds to obtained amplified alginate-specific DNA aptamers having higher affinity for alginate following each round.

It should be understood that each round of incubating an alginate-specific DNA aptamer with alginate hydrogel to collection of the alginate-specific DNA aptamer results in a selection of an alginate-specific DNA aptamer. Following amplification of the collected alginate-specific DNA aptamer and subsequent incubation with an alginate hydrogel, the newly collected alginate-specific DNA aptamer is expected to have increased affinity for alginate.

Suitable amount of alginate in the final alginate hydrogel composition ranges from about 0.05% (wt/wt) to about 10% (wt/wt). Preferably, the amount of alginate ranges from about 0.5% (wt/wt) to about 2% (wt/wt).

A suitable hydrogel degradation buffer includes 1× PBS, 100 mM EDTA, and 2 M urea. A suitable alginate lyase solution includes 1.5 U of alginate lyase in 20 mM MES pH 4.5. Suitably, the incompletely degraded alginate hydrogel fragments can be removed from the sample using an Amicon 15K centrifugal column. It should be understood that degrading the alginate hydrogel need not degrade the alginate hydrogel to monomers. Other suitable methods for degrading the alginate hydrogel include incubating the alginate hydrogel in a solution of EDTA, incubating the alginate solution in phosphate buffered saline and applying heat.

The method further includes analyzing the alginate-specific DNA aptamers. The alginate-specific DNA aptamers can be analyzed by can be amplification such as PCR. The alginate-specific DNA aptamers can also be cloned and sequenced.

In another aspect, the present disclosure is directed to a method for delivering to a subject in need thereof a biomolecule in an alginate hydrogel. The method includes: providing to a subject in need thereof an alginate hydrogel, the alginate hydrogel having a biomolecule-alginate-specific DNA aptamer conjugate, wherein the biomolecule-alginate-specific DNA aptamer conjugate specifically binds to alginate by the alginate-specific DNA aptamer and wherein the biomolecule-alginate-specific DNA aptamer conjugate is released from the alginate hydrogel to deliver the biomolecule.

The method includes conjugating a biomolecule to an alginate-specific DNA aptamer. The biomolecule is conjugated to the alginate-specific DNA aptamer to form a biomolecule-alginate-specific DNA aptamer complex, which is then incorporated into an alginate hydrogel. The biomolecule is conjugated to the alginate-specific DNA aptamer using methods known in the art. For example, a protein is conjugated to a 5′ amine-modified alginate-specific DNA aptamer in 25 mM MES pH 6.0 and 5 mg/mL EDC. Unreacted reagents are removed and the conjugated protein-alginate-specific DNA aptamer are purified. For example, the conjugated protein-alginate-specific DNA aptamer is purified by passing the conjugation reaction through SEPHACRYL S-100 in a spin column, eluted from the column by adding 50 μL of 50 mM Na₂PO₄ PH 7.2 and 150 mM NaCl and centrifuging at 500 rpm for 2 minutes. Column fractions can be monitored for protein by measuring the A280 and confirmed via SDS-PAGE.

The conjugated biomolecule-alginate-specific DNA aptamer is then loaded into the alginate hydrogel. Loading of the conjugated biomolecule-alginate-specific DNA aptamer into the alginate hydrogel is by reconstituting the conjugated biomolecule-alginate-specific DNA aptamer in an alginate solution and initiating gelation of the alginate solution to form an alginate hydrogel. Alternatively, the conjugated biomolecule-alginate-specific DNA aptamer in solution is incubated with a preformed alginate hydrogel.

The amount of alginate in the final alginate hydrogel composition ranges from about 0.05% (wt/wt) to about 10% (wt/wt). Preferably, the amount of alginate ranges from about 0.5% (wt/wt) to about 2% (wt/wt).

Any biomolecule of interest can be conjugated to the alginate-specific DNA aptamer. Suitable biomolecules include nucleic acids, small molecule drugs, proteins, peptides, liposomes, viruses, bacteria, cells, and combinations thereof (as described in He et al. Acta Pharm Sin B. 2023 April; 13(4): 1358-1370, which is incorporated by reference herein). For example, the alginate-specific DNA aptamer and drug can form covalent coupling by modifying a reactive group, such as amino, thiol, cyclooctyne (DBCO), thiol-maleimide, azide-alkyne, and amino-carboxyl methods. Other methods include solid-phase synthesis, enzymatic coupling, nucleic acid complementary hybridization (as described in He et al. Acta Pharm Sin B. 2023 April; 13(4): 1358-1370, which is incorporated by reference herein).

Suitable subjects include animals. Particularly suitable subjects are humans, primates, mice, rats, rabbits, birds, and other animals. Suitable subjects include animals suffering from a disease or disorder that would require administration of a biomolecule such as a therapeutic agent.

In another aspect, the present disclosure is directed to a method for analyzing small molecule drug binding to serum albumin. The method includes coupling serum albumin to an alginate-specific DNA aptamer to form an alginate-specific DNA aptamer-serum albumin conjugate; encapsulating the alginate-specific DNA aptamer-serum albumin conjugate within an alginate hydrogel; incubating a small molecule drug with the alginate hydrogel; washing the alginate hydrogel; collecting the wash solution; and analyzing the wash solution to determine a small molecule drug-serum albumin binding profile.

The term “small molecule drug” is used according to its ordinary meaning as understood by one of ordinary skill in the art to refer to a low molecular weight (≤1000 daltons) organic compound that may regulate a biological process.

Washing the alginate hydrogel after incubating the small molecule drug with the alginate hydrogel can be repeated for as many times as desired and each wash solution is collected after each washing step. An amount of the small molecule drug can be determined by high pressure liquid chromatography (HPLC).

The method can further include degrading the alginate hydrogel. The alginate hydrogel can be degraded by chelation using a chelating solution (e.g., 0.5 M EDTA solution).

An amount of the small molecule drug can be determined after degrading the alginate hydrogel by high pressure liquid chromatography (HPLC).

Suitable serum albumin is human serum albumin (HSA) and bovine serum albumin. In a particularly suitable embodiment, the serum albumin is human serum albumin (HSA).

Serum albumin can be coupled with an alginate-specific DNA aptamer using known methods. For example, the coupling agent DMT-MM can be used to couple serum albumin with an alginate-specific DNA aptamer.

The alginate hydrogel including serum albumin conjugated to an alginate-specific DNA aptamer of the method is prepared by conjugating the serum albumin to the alginate-specific DNA aptamer. The aptamer-bound serum albumin is then added to an alginate precursor solution. Gelation of the precursor solution to form the alginate hydrogel occurs upon addition of a crosslinking agent (e.g., CaCl₂).

The amount of alginate in the final alginate hydrogel composition ranges from about 0.05% (wt/wt) to about 10% (wt/wt). Preferably, the amount of alginate ranges from about 0.5% (wt/wt) to about 2% (wt/wt).

### EXAMPLES

### Example 1

### Materials and Methods

Materials: DNA oligonucleotides were synthesized by Integrated DNA Technologies (IDT, Coralville, IA) and purified either via denaturing polyacrylamide gel (PAGE) or phenol: chloroform: isoamyl alcohol (PCI) extraction. Alginic acid sodium salt and glucono-1,5-lactone were purchased from RPI (Mt. Pleasant, IL). EDC (1-ethyl-3-(3-dimethylaminopropyl) carbodiimide hydrochloride), tetramethylrhodamine (TAMRA) NHS ester and Pierce centrifuge columns were purchased from Thermo Fisher (Waltham, MA). Agarose, alginate lyase, Amicon Ultra 15 centrifugal filter units, phenol:chloroform:isoamyl alcohol 25:24:1 saturated with 10 mM Tris, pH 8.0 and 1 mM EDTA, and SEPHACRYL S-100HR were from Millipore Sigma (St. Louis, MO). Bovine serum albumin (BSA) was purchased from New England Biolabs (NEB, Ipswich,MA). 72-well culture plates were purchased from Biologix (Camarillo,CA).

In vitro Selection: In vitro selection (FIG. 1) was initiated by reconstituting 0.3 nmol of the appropriate DNA oligonucleotide pool in a 1% alginate solution to a volume of 400 μL. NaCl, CaCl₂, and glucono-1,5-lactone were added and the solution was pipetted into empty spin columns and allowed to gel for 30 min. The final hydrogels containing the DNA pools were 1% alginate, 30 mM NaCl, 30 mM CaCl₂, and 120 mM glucono-1,5-lactone. Two different pools were used, with either 40 or 60 randomized nucleotides flanked by fixed primer binding regions. A total of 4 selections, designated with two letters as shown in FIG. 1, were set up to test each randomized pool with 1× PBS (pH 7.4) containing either 10 mM CaCl₂ or 10 mM MgCl₂. The pool-containing hydrogels were incubated in appropriate selection buffer overnight at room temperature. The buffer was exchanged by centrifuging the spin columns at 2200 rpm for 15 min and the flowthrough was collected. 400 μL of appropriate selection buffer was added to each hydrogel and the samples were centrifuged as above, with the flowthrough again collected as washes. A total of 3 washes were conducted. Oligonucleotides that bound the alginate were recovered by degrading the hydrogel in 1× PBS, 100 mM EDTA, and 2 M urea at 55° C. for 15 min, followed by treatment with 1.5 U of alginate lyase in 20 mM MES pH 4.5 at 37° C. overnight. After alginate lyase treatment, any incompletely degraded gel fragments were removed from the sample using an Amicon 15K centrifugal column. The recovered sequences were ethanol precipitated prior to PCR.

Recovered oligonucleotides were amplified by PCR using forward primer 5′-GAACTAGATCGCAGC-3′ (SEQ ID NO:5) and reverse primer 5′-CAACAACAACAA\hat{ }GGATTACCTCGATCC-3′ (SEQ ID NO:6), where “\hat{ }” located between nucleotides 12 and 13 represents a non-amplifiable linker that allows for strand separation on denaturing PAGE to regenerate the desired single-stranded pool. 10 cycles of PCR were followed by 30 cycles of PCR using 5′ TAMRA-labeled forward primer in a ratio of 1:24 with unlabeled forward primer to add a fluorescent tag to the desired isolated sequence strand. This labeled primer was generated by conjugating TAMRA-NHS to 5′ amine-modified forward primer. The desired single-stranded DNA product was isolated via denaturing PAGE and extracted from the gel as previously described for use in the next selection round. The selection progress was then monitored by measuring fluorescence in the collected fractions using a plate reader. The excitation wavelength was set at 540 nm and the emission was collected at 590 nm with a scan rate of 4 ms.

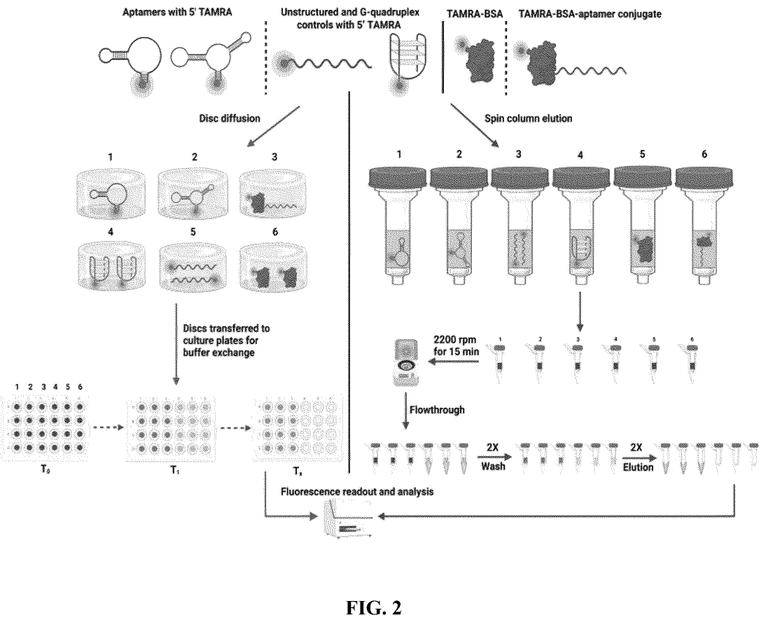

To ensure isolated sequences were not an artifact of our spin column approach and to apply a selection pressure for delayed release from the hydrogels, we incorporated a disc diffusion step after round 5. Cylindrical alginate hydrogel discs were fabricated with encapsulated pool sequences in molds to a volume of 100 μL (FIG. 2). Once the gelation was complete, the discs were placed in individual culture plate wells and were covered with 200 μL of appropriate selection buffer for overnight incubation. The disc test was done in triplicate for each selection, along with two control sequences: a G-quadruplex 5′-GGGTCATTGTGGGTGGGTGTGGG-3′ (SEQ ID NO:7) and a sequence expected to be unstructured 5′-CAACAACAACAACAACAACAACAACAACAACAACAACAACAACAACAAC AACAACAACAACAACAACAAC-3′ (SEQ ID NO:8). Buffer exchange was performed at 0, 2, 4, 6, 8, 12, and 24 h post initial overnight incubation. The discs were degraded as described above after 26 h. After measuring the fluorescence of the collected elutions, sequences from the 12, 24, and 26 h time points were combined, ethanol precipitated, and PCR amplified for the next round of selection. This disc diffusion assay was repeated in round 7 to assess the selections prior to cloning.

Initial aptamer characterization: Sequences recovered after round 7 were prepared for cloning via PCR using forward primer 5′-TAATTAATTAATTAGAACTAGATCGCAGC-3′ (SEQ ID NO:9) and reverse primer 5′-TAATTAATTAATTAGGATTACCTCGATCC-3′ (SEQ ID NO:10), which contain stop codons to reduce false positives in blue-white screening. The resulting products were purified by agarose gel and cloned using a TOPO TA Cloning Kit (Invitrogen, Carlsbad, CA). Clones were checked for inserts using colony PCR and plasmids from positive clones were isolated via spin minipreps (IBI Scientific, Dubuque, IA). These plasmids served as the template for PCR to generate individual aptamers for an initial screen using the same spin column approach used in selections above. Aptamers with the best elution profiles were sequenced (ACGT, Inc., Wheeling, IL) and synthesized by IDT with a 5′ NH₂ to allow for labeling with TAMRA-NHS ester.

Identifying the most promising aptamer candidates: Labeled aptamers were tested via disc diffusion to identify those sequences with the best alginate binding properties (FIG. 2). Buffer exchange was expanded from one day out to one week prior to final gel degradation. The spin column method was also repeated to confirm alginate binding. For the best binding aptamers, diffusion was measured in discs made from 0.5%, 1%, or 2% alginate.

Aptamer binding to prefabricated hydrogels: To rule out that the aptamers were simply trapped in the hydrogel, alginate hydrogels, in both disc and spin column format, were fabricated without the reconstituted aptamers. Labeled aptamers were folded by denaturation at 95° C. for 3 min and annealing on ice for 5 mins and then were introduced to the hydrogels in appropriate buffer.

Assessing diffusion of aptamer-conjugated BSA: To access the diffusion profile of identified aptamers with conjugated biomolecules of interest, 2 mg BSA was first labeled with TAMRA-NHS ester in 1M CHES pH 9.1 and 5 mM EDTA for 5 h. The labeled BSA was then conjugated to 5′ amine-modified aptamer in 25 mM MES pH 6.0 and 5 mg/mL EDC and reaction was allowed to run overnight at 4° C. Unreacted reagents were removed by passing the reaction through SEPHACRYL S-100 in a spin column. Samples were eluted from the column by adding 50 μL of 50 mM Na₂PO₄ PH 7.2 and 150 mM NaCl and centrifuging at 500 rpm for 2 min. Fractions were monitored for protein by measuring the A280 and confirmed via SDS PAGE. 40 μg of TAMRA-labeled aptamer-BSA conjugates were encapsulated in 1% alginate hydrogel in either a disc for diffusion tests or a spin column for elution tests (FIG. 2).

### Results and Discussion

SELEX for alginate-binding aptamers and preliminary binding tests: To isolate aptamers that bind to alginate hydrogels, we chose to use the hydrogels themselves as targets, rather than using monomers or small oligomers of the alginate building blocks. By using hydrogel formed from readily available commercial alginate, the potential aptamers are presented with different binding sites as alginate hydrogels can have regions predominately composed of G (G block), M (M block), or mixed G and M. Thus the collection of isolated aptamers may contain some aptamers that are specific for one of these blocks. As different formulations of alginate can have different ratios of these blocks, having aptamers for the different blocks could be advantageous, but are not expected to be necessary in many uses.

To facilitate this selection approach, DNA pools were encapsulated during hydrogel preparation and the hydrogels were allowed to form within empty spin columns. Preliminary diffusion studies showed that the hydrogels were swelling during the first hours of testing. This swelling is expected to change the internal hydrogel structures that the aptamers could bind, so fabricated hydrogels with encapsulated pool were incubated for 24 h prior to beginning the wash and elution steps. This incubation allowed the gels to fully swell while providing the candidate aptamers ample time to equilibrate and establish binding. By utilizing the spin column setup and a slow centrifuge speed, we were able to fully elute the candidate aptamers better than a simple diffusion approach without compressing the hydrogel.

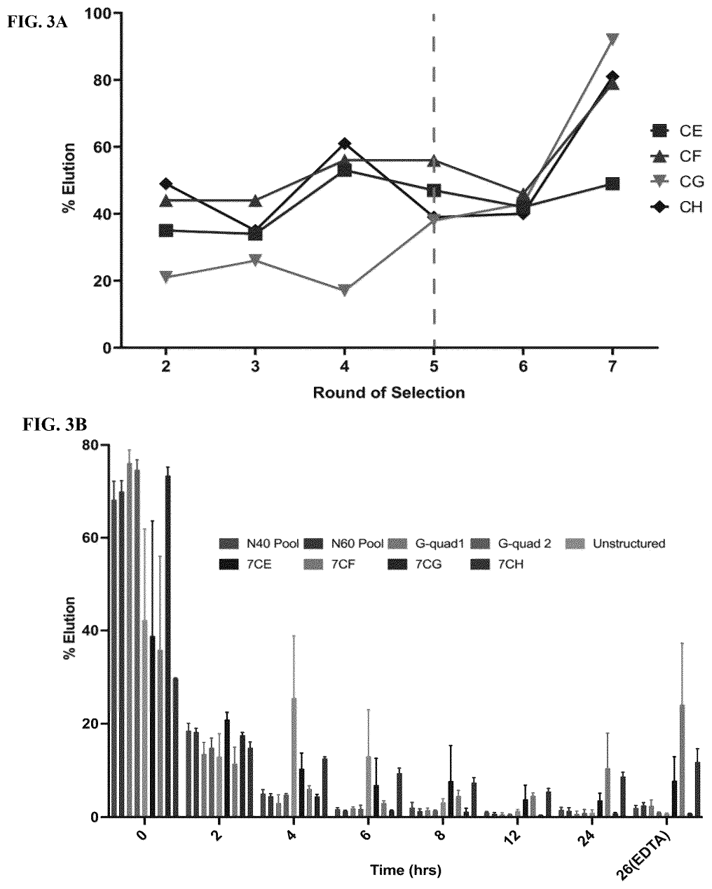

As shown in FIG. 3A, a slight increase in eluted sequences was observed for most of the selection conditions in round 4. To assess if the observed elution profile was due to alginate binding, the recovered sequences from round 4 were amplified via PCR and encapsulated in hydrogels for a disc diffusion assay lasting up to 24 hours. Except for selection CE, delayed release of at least a subset of candidate aptamers was observed. The eluted sequences from time points greater than 12 hours were recovered, amplified by PCR, and used at the candidate sequences for round 5. At round 7, selections CF, CG, and CH showed substantial increases in elution. The eluted sequences from round 7 for each of the selections was analyzed for binding using the disc diffusion test (FIG. 3B). For all tested sequences, including controls, a large amount of release was detected with the first buffer exchange (0 h, FIG. 3B) after the initial 24 h incubation. This initial burst release from alginate hydrogels has been demonstrated extensively for proteins and other biomolecules. There was a bimodal release of aptamers from the hydrogel discs. The initial burst, while high, was less than that observed with controls and was followed by a slow, measurable release over time. Additionally, 7CF and 7CH showed higher release at 24 h and when the gel was fully degraded, indicating these sequences were held in the hydrogel more effectively. Based on these results, the recovered oligonucleotides from round 7 for CF and CH were cloned and sequenced. Analysis of the resultant aptamer sequences revealed some overall changes in the lengths of the resulting aptamers. While the starting aptamer pool were 100 nt for the CF and CH selections (N60 random region plus primer binding sites), some nucleotide insertions and deletions occurred, which is not considered uncommon. The resulting sequences were also compared to assess homology and to identify unique sequences.

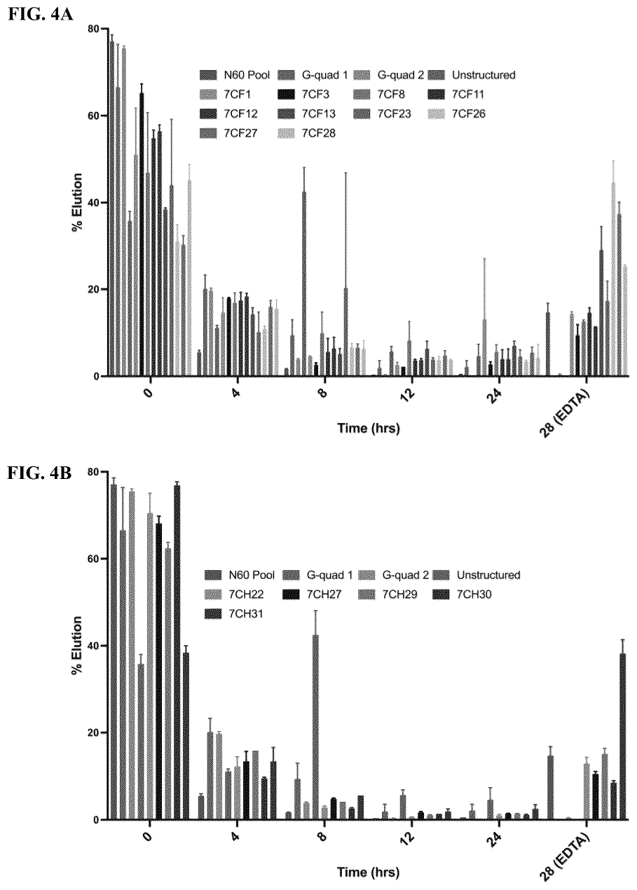

Unique sequences from 7CF and 7CH were initially analyzed via disc diffusion (FIG. 4). For the 7CF clones, 7CF13 (SEQ ID NO:1), 7CF26 (SEQ ID NO:3), and 7CF27 (SEQ ID NO:4) were retained longer in the hydrogel than controls and produced a strong spike when the hydrogel was degraded completely (FIG. 4A). Similarly for the 7CH clones, 7CH31 (SEQ ID NO:2) produced strong spikes when the hydrogel was degraded (FIG. 4B). These aptamers were further tested.

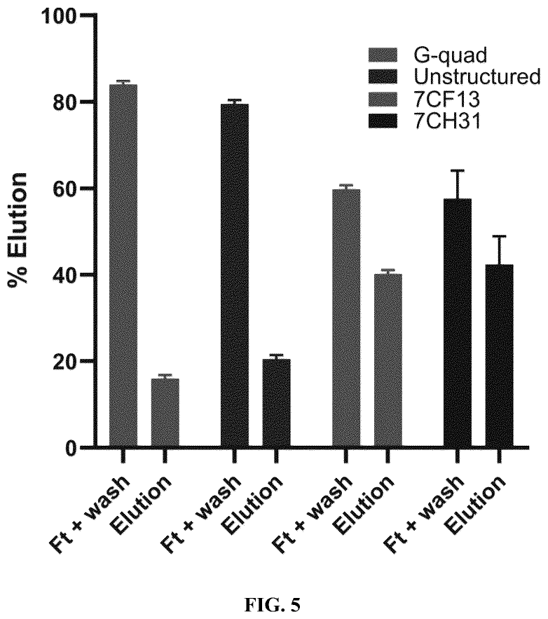

The disc diffusion assay represents an intended application for alginate aptamers in which the aptamers or molecules conjugated to the aptamers are loaded into hydrogels during formation and are then slowly released. The binding profile of the aptamers was confirmed in the spin column elution approach used during SELEX (FIG. 5). For controls, no more than 20% of the oligonucleotides were in the recovered elution. At least 40% of aptamers 7CF13 and 7CH31 were recovered during elution, providing additional evidence that the aptamers bind tighter to alginate hydrogels than control oligonucleotides.

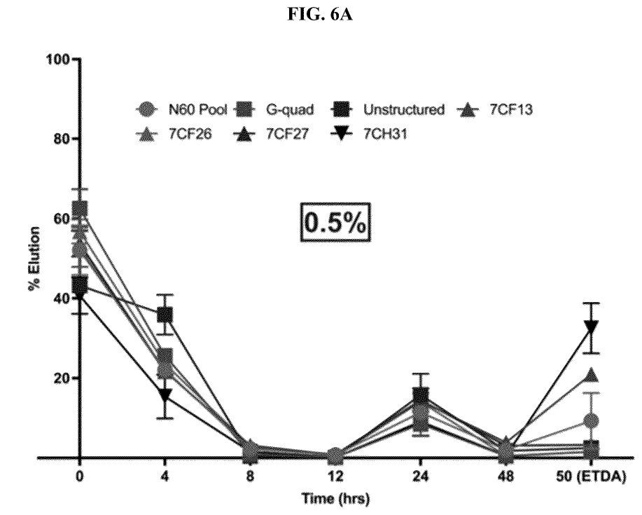

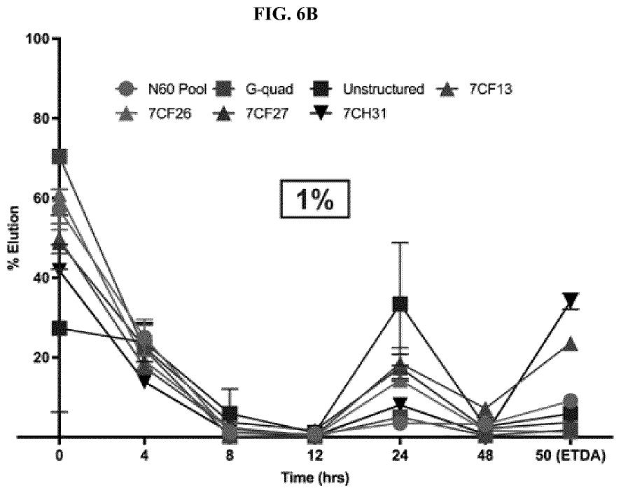

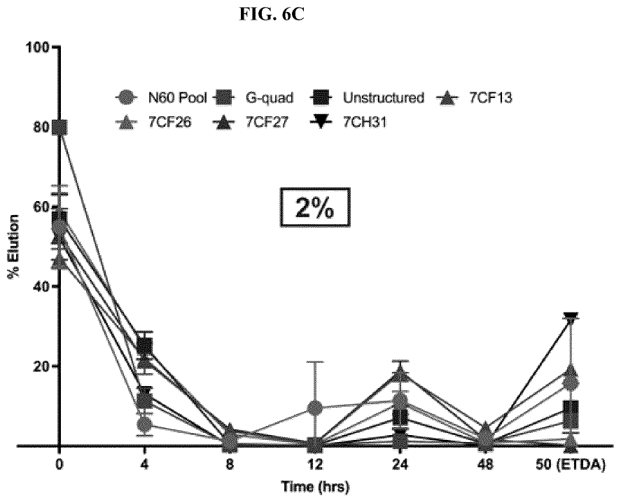

Hydrogels made with 1% alginate are commonly used in applications, so that was the percentage used during SELEX. Different percentages of alginate can impact hydrogel properties, which could also affect aptamer binding. Our best candidate aptamers were encapsulated in either 0.5%, 1%, or 2% alginate hydrogels and allowed to diffuse out of the gels for up to 50 h. While we observed a spike in release at the 24 h time point for all tested oligonucleotides, the aptamers showed delayed release relative to controls for all percentages (FIG. 6). Importantly, two aptamers, 7CF13 and 7CH31, showed substantial release upon hydrogel degradation after 50 h. A 2.5-fold increase in elution upon degradation was observed for the aptamers compared to controls. These two sequences were selected for further testing.

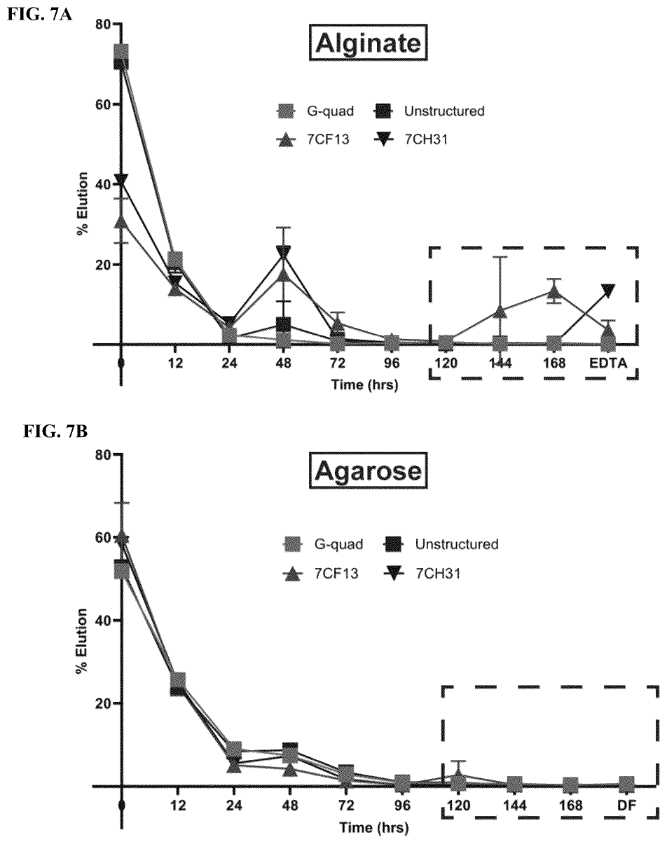

Specificity for alginate: To test the specificity of the aptamers for alginate, aptamers and controls were encapsulated in 1% agarose discs. Agarose represents a different polysaccharide that also forms porous gels. Diffusion of the aptamers out of the agarose discs was monitored for over one week (FIG. 7). Aptamers 7CF13 and 7CH31 were both confirmed to be specific binders for alginate hydrogels, as shown by higher retention in the alginate hydrogel after the initial burst release, a secondary spike at 48 h and substantial release upon degrading the alginate hydrogel (FIG. 7A). For agarose gels, the aptamers and controls showed the same diffusion profiles with little oligonucleotide remaining in the agarose gel after 120 h (FIG. 7B).

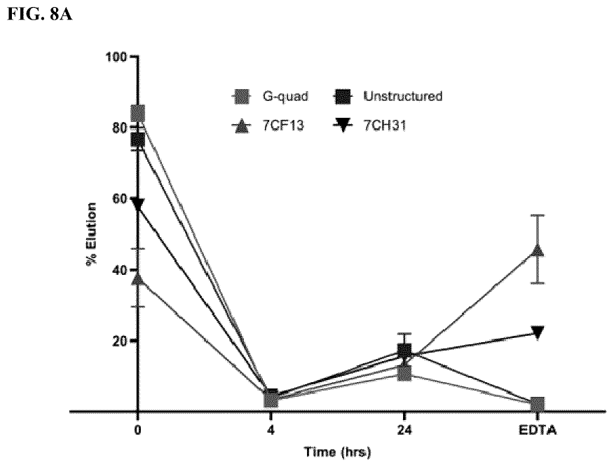

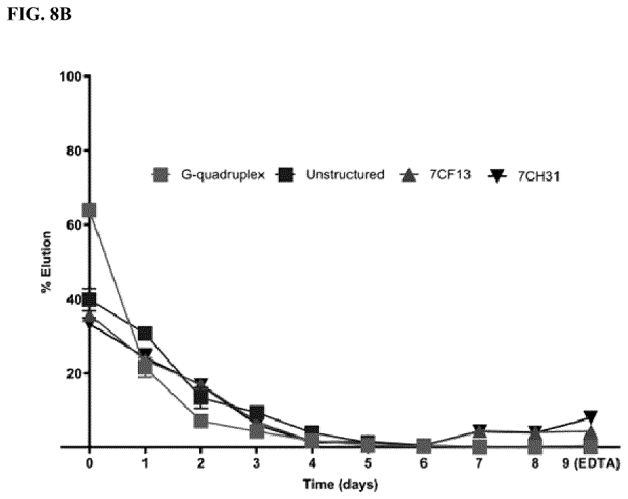

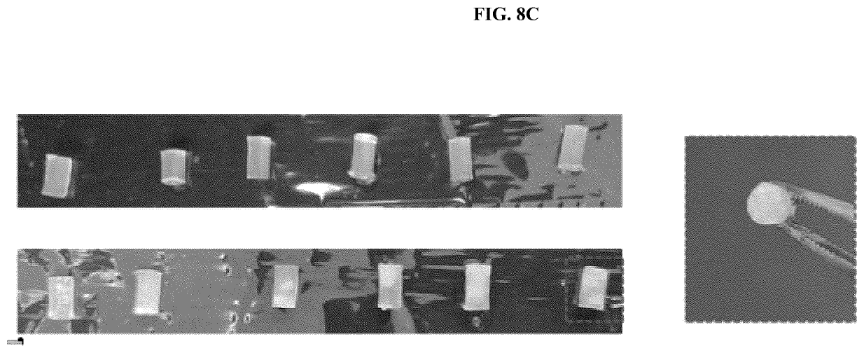

Binding of aptamers to preformed alginate hydrogel discs: In the selection and the subsequent characterization tests, oligonucleotides (aptamers and controls) were encapsulated during hydrogel formation and allowed to incubate overnight in buffer to allow hydrogel swelling. To ensure that the observed aptamer binding was not dependent on entrapment of aptamers, hydrogel discs were first fabricated without the aptamers. TAMRA-labeled aptamers were then introduced to the discs in binding buffer and incubated overnight to allow the aptamers to diffuse into the hydrogel. Buffer exchange was done at regular time points and the hydrogel discs were degraded after 24 h (FIG. 8A). Aptamers 7CF13 and 7CH31 were able to bind to preformed hydrogel disc better than the controls, with ˜80% of the control oligonucleotides being recovered during the initial buffer exchange compared to 60% for 7CH31 and 40% for 7CF13. Once bound, the aptamers displayed similar diffusion to encapsulated aptamers, with increased release after 7 days and upon gel degradation (FIG. 8B).

Closer visualization of the hydrogels with the introduced oligonucleotides also revealed an interesting observation. Nucleation was observed in discs containing 7CF13 and 7CH31, more pronounced nucleation for aptamer 7CH31 (FIG. 8C). The nucleation sites more likely represent the areas in the hydrogel discs with ideal binding pockets for the identified aptamers. Once the aptamers are introduced to the preformed hydrogel discs, the TAMRA labeled aptamers swarmed to these sites. These results demonstrate that aptamer binding to alginate hydrogels can be achieved regardless of when the aptamers are introduced.

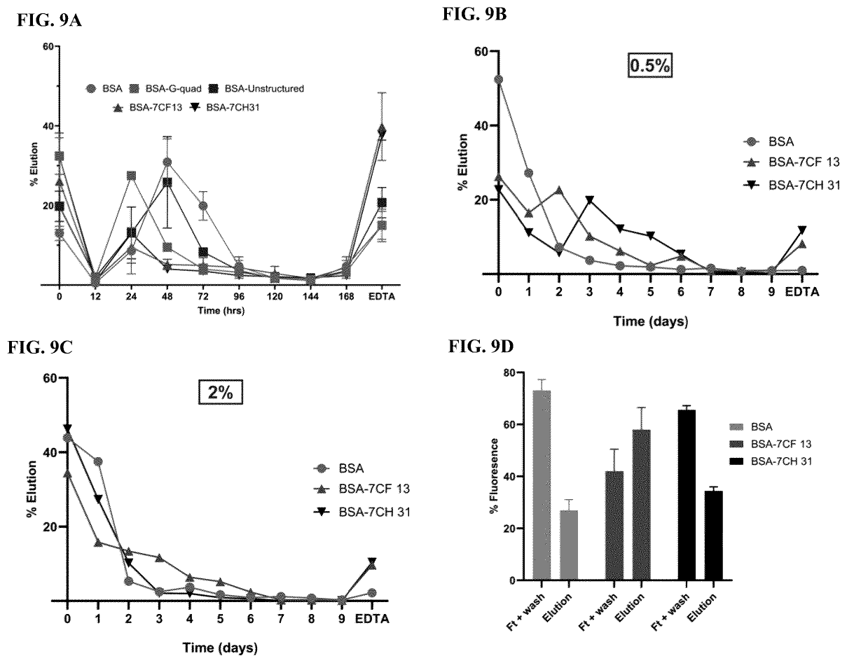

Ability of alginate aptamers to impact diffusion of a conjugated protein: One motivation for identifying aptamers for alginate hydrogels was the potential ability to control the diffusion of other molecules from hydrogels. To assess this ability for our identified aptamers, we conjugated each aptamer to TAMRA-labeled BSA. The diffusion profile of BSA out of 1% hydrogel discs has already been demonstrated,⁵ so our initial assay involved diffusion of encapsulated BSA conjugated to control oligonucleotides or our aptamers (FIG. 9A). Following an initial burst release, differences in diffusion profiles start to emerge. The G-quadruplex control conjugate shows a secondary spike at the 24 h mark, while unconjugated BSA and unstructured control conjugates peak at 48 h. For the aptamer conjugates, both had a small spike at 24 h mark but were held more tightly in the hydrogel showed a larger, more significant spike when the hydrogel was degraded after 7 days.

Based on these results, we tested the ability of the aptamer-BSA conjugates to enter and then be retained by preformed alginate discs, using two different percentages of alginate to prepare the discs. When comparing unconjugated BSA to aptamer-conjugated BSA, the unconjugated BSA was generally not initially captured as well by the hydrogel (as indicated by a high degree of elution at 0 h), diffused out faster, and was mostly released by day 4 (FIGS. 9B-C). The aptamer-conjugated BSA was better captured by the hydrogel discs, particularly at lower alginate percentages. The aptamer conjugates also diffused out more slowly over 9 days. Approximately 10% of each of the aptamer conjugates was released when the discs were degraded after Day 9, compared to 2% of the unconjugated BSA, representing a 5-fold improvement in retention. Additionally, we also encapsulated BSA and aptamer-conjugated BSA in hydrogels within spin columns to confirm the superior retention of the aptamer conjugates. Once again, the aptamer conjugated-BSA was bound more tightly than BSA alone, with aptamer 7CF13 providing better retention prior to elution (FIG. 9D). The results demonstrate that the alginate aptamers can impact the diffusion of conjugated biomolecules and can extend the release of biomolecules from alginate hydrogels when conjugated to the alginate-specific aptamers.

The alginate-specific DNA aptamers of the present disclosure consistently showed better binding and diffusion profiles to alginate hydrogels than the control sequences in a variety of tests to gauge alginate specificity and applicability in the clinical setting by loading alginate-specific DNA aptamers conjugated to a representative biomolecule (BSA). Binding of the alginate-specific DNA aptamers to preformed alginate hydrogels or encapsulating them into alginate hydrogels prior to gelation allows for flexibility in the clinical setting where a therapeutic of choice is preloaded in alginate hydrogels or its dosage can be adjusted later to meet patient specific needs.

### Example 2

### Materials and Methods

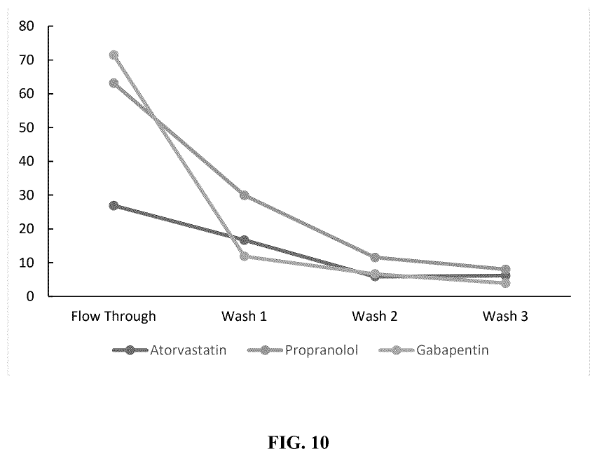

The DNA aptamer with the sequence GCAAGATCACGAGTCAGTCACTGGGGCTCTACGGCAGGCGATAATCCATC TTCCATAGGGGGAGCAGTCCCGTGGGTACACTAGATCGCAGTAGCTGATC (SEQ ID NO:1) has been previously conjugated to BSA and demonstrated the ability to retain BSA within a 1% alginate hydrogel for at least one week. 10 nanomoles of the 5′-NH2 version of the DNA aptamer (SEQ ID NO:1) was conjugated to HSA using the coupling agent DMT-MM. The resultant complex was purified via size-exclusion chromatography. Subsequently, 20 μL of DNA aptamer-bound HSA was added to a 1% alginate solution and a hydrogel disc was formed by the addition of 30 mM CaCl₂ and 120 mM delta gluconolactone. 1 mM of the desired drug (Atorvastin, Propranolol and Gabapentin) was introduced to 10 mM aptamer-tethered HSA in 1% alginate hydrogel discs, followed by multiple wash steps. The hydrogels were then degraded via chelation with a 0.5 M EDTA solution. The quantity of drug in each wash and after degradation was quantified using HPLC. As shown in FIG. 10, different binding profiles were observed for these drugs.

The aptamer-tethered HSA in alginate hydrogel provide a stable testing environment to investigate the binding of drugs to HSA. This interaction can inform therapeutic decision-making and drug design. Not only can measurements be made on the amount of drug captured by the bound HSA, but if desired, the HSA could later be released to isolate the HSA-drug complexes.

Predictive models that assess drug-HSA interactions are crucial for drug development. These models, which use both structure-and ligand-based approaches, aid in screening drug candidates and optimizing lead compounds. By predicting the binding affinities and understanding the molecular dynamics of drug-HSA interactions, researchers can design drugs with optimal absorption, distribution, metabolism, and excretion (ADME) profiles.

The alginate-binding aptamers of the present disclosure allow for immobilizing HSA within a stable testing environment (alginate hydrogels) for binding studies. Not only can measurements be made on the amount of drug captured by the bound HSA, but if desired, the HSA can be released later to isolate the HSA-drug complexes.

The present disclosure provides alginate-specific DNA aptamers and alginate hydrogels having alginate-specific DNA aptamers. The present disclosure also provides a method for selecting alginate-specific DNA aptamers. The alginate-specific DNA aptamers provide a new method to deliver a biomolecule in an alginate hydrogel to provide controlled release of biomolecules conjugated to an alginate specific-DNA aptamer.

What is claimed is:

1. An alginate-specific deoxyribonucleic acid (DNA) aptamer.

2. The alginate-specific deoxyribonucleic acid (DNA) aptamer of claim 1, wherein the alginate-specific deoxyribonucleic acid (DNA) aptamer is conjugated to serum albumin.

3. The alginate-specific deoxyribonucleic acid (DNA) aptamer of claim 2, wherein the serum albumin is selected from the group consisting of human serum albumin, bovine serum albumin, and combinations thereof.

4. The alginate-specific deoxyribonucleic acid (DNA) aptamer of claim 1, ranging in length from about 10 nucleotides to about 250 nucleotides.

5. The alginate-specific deoxyribonucleic acid (DNA) aptamer of claim 1, further comprising a primer binding site.

6. The alginate-specific deoxyribonucleic acid (DNA) aptamer of claim 1, further comprising a label.

7. An alginate hydrogel comprising alginate and an alginate-specific deoxyribonucleic acid (DNA) aptamer.

8. The alginate hydrogel of claim 7, wherein the alginate-specific DNA aptamer is conjugated to serum albumin.

9. The alginate hydrogel of claim 8, wherein the serum albumin is selected from the group consisting of human serum albumin, bovine serum albumin, and combinations thereof.

10. The alginate hydrogel of claim 7, wherein the alginate in the alginate hydrogel ranges from about 0.05% (wt/wt) alginate to about 10% (wt/wt) alginate.

11. A method for selecting an alginate-specific deoxyribonucleic acid (DNA) aptamer, the method comprising:
(a) reconstituting a deoxyribonucleic acid (DNA) oligonucleotide mixture in an alginate solution;
(b) initiating gelation of the alginate solution to form an alginate hydrogel including the DNA oligonucleotide mixture;
(c) incubating the alginate hydrogel in a selection buffer;
(d) collecting the selection buffer;
(e) degrading the alginate hydrogel to form a degradation solution;
(f) treating the degradation solution with an alginate lyase solution;
(g) collecting the alginate-specific DNA aptamers from at least one of the selection buffer, the degradation solution, the alginate lyase solution, and combinations thereof; and
(h) amplifying the alginate-specific DNA aptamers.

12. The method of claim 11, further comprising: repeating step (a)-step (h) at least once.

13. A method for delivering to a subject in need thereof a biomolecule in an alginate hydrogel, the method comprising:
providing to a subject in need thereof an alginate hydrogel, the alginate hydrogel comprising a biomolecule-alginate-specific deoxyribonucleic acid (DNA) aptamer conjugate, wherein the biomolecule-alginate-specific DNA aptamer conjugate specifically binds to alginate by the alginate-specific DNA aptamer and wherein the biomolecule-alginate-specific DNA aptamer conjugate is released from the alginate hydrogel to deliver the biomolecule.

14. A method of analyzing binding of a small molecule drug to human serum albumin, the method comprising:
contacting a small molecule drug with an alginate hydrogel, the alginate hydrogel comprising an alginate-specific DNA aptamer conjugated with human serum albumin;
washing the alginate hydrogel with a wash solution; and
analyzing the wash solution to determine binding of the small molecule drug to the human serum albumin.

15. The method of claim 14, further comprising degrading the alginate hydrogel with a degradation solution and analyzing the degradation solution to determine binding of the small molecule drug to the human serum albumin.


SEQUENCE LISTING (10 sequences)

SEQ ID NO: 1 - DNA, 100 bp, synthetic construct
  source 1..100: mol_type = other DNA
gcaagatcacgagtcagtcactggggctctacggcaggcgataatccatcttccatagggggagcagtcccgtgggtacactagatcgcagtagctgatc

SEQ ID NO: 2 - DNA, 100 bp, synthetic construct
  source 1..100: mol_type = other DNA
gcaagatcacgagtcagtagacctatgctgtagtcggacgatcagagccgacctgagagagccggtaggtgggatatacactagatcgcagtagctgatc

SEQ ID NO: 3 - DNA, 100 bp, synthetic construct
  source 1..100: mol_type = other DNA
gcaagatcacgagtcagtagaaaagccggcccatgcctattcgttgtcaccgttgacttctcccgtgacgcataatcccactagatcgcagtagctgatc

SEQ ID NO: 4 - DNA, 100 bp, synthetic construct
  source 1..100: mol_type = other DNA
gcaagatcacgagtcagtacgatgggggttgcatacaggatcctctcaatgagtcccatagatactccggttaacagtcactagatcgcagtagctgatc

SEQ ID NO: 5 - DNA, 15 bp, synthetic construct
  source 1..15: mol_type = other DNA
gaactagatcgcagc

SEQ ID NO: 6 - DNA, 27 bp, synthetic construct
  source 1..27: mol_type = other DNA
caacaacaacaaggattacctcgatcc

SEQ ID NO: 7 - skipped

SEQ ID NO: 8 - DNA, 23 bp, synthetic construct
  source 1..23: mol_type = other DNA
gggtcattgtgggtgggtgtggg

SEQ ID NO: 9 - DNA, 70 bp, synthetic construct
  source 1..70: mol_type = other DNA
caacaacaacaacaacaacaacaacaacaacaacaacaacaacaacaacaacaacaacaacaacaacaac

SEQ ID NO: 10 - DNA, 29 bp, synthetic construct
  source 1..29: mol_type = other DNA
taattaattaattagaactagatcgcagc